(19) United States
(12) Patent Application Publication
(10) Pub. No.: US 2004/0133169 A1
(43) Pub. Date: Jul. 8, 2004

(54) Syringe cylinder

(75) Inventors: Jochen Heinz, Kiel (DE); Dieter Schilling, Aukrug-Innien (DE)

Correspondence Address: COHEN, PONTANI, LIEBERMAN & PAVANE, 551 FIFTH AVENUE, SUITE 1210, NEW YORK, NY, 10176, US

(73) Assignee: Transcoject Gesellschaft fur medizinische Gerate mbH & Co. KG

(21) Appl. No.: 10/684,886

(22) Filed: Oct. 14, 2003

(30) Foreign Application Priority Data
Oct. 15, 2002 (DE) 202 15 807.1

(51) Int. Cl. A61M 5/00

(52) U.S. Cl. 604/187

(57) ABSTRACT

A syringe cylinder is open to one side for accommodating a plunger and at the other side comprises a syringe connection in the form of a Luer lock connection. The Luer lock connection is formed by a Luer connection and a cylinder section with an inner thread, the cylinder section surrounding the Luer connection. On the outer circumference of the cylinder section there is arranged a ring tapering towards the connection-side end.

### BACKGROUND OF THE INVENTION

1. Field of the Invention

The invention relates to a syringe cylinder, in particular for a prefillable or prefilled syringe having an open end for receiving a plunger and an opposed end with a syringe connection in the form of a Luer lock connection.

2. Description of the Related Art

Syringe cylinders of this type are manufactured of glass or in the meantime also of plastic and typically comprise a Luer connection which holds the closure plug with which the prefilled syringe cylinder is closed, and to which a cannula, flexible tubing or likewise may be connected after removal of the closure plug. Such a syringe cylinder is for example known from U.S. Pat. No. 5,624,402.

The disadvantage of these syringe cylinders provided with a Luer connection is the fact that they may only be connected easily and in an uncomplicated manner to connection systems compatible with the Luer or Luer lock. There often arises however the problem that one for example should connect a flexible tubing on which such a connection is not present. The assembly and sealing then becomes quite complicated.

### SUMMARY OF THE INVENTION

Against this background it is the object of the invention to provide a syringe cylinder with a Luer connection which is inexpensive to manufacture and may be applied in a comprehensive as possible manner.

The basic concept of the present invention is to provide means on the outer side of the Luer connection which permit a flexible tubing to be connected directly and which in particular simplifies the sliding on and the sealing. For this the invention envisages at least one or also more rings arranged behind one another and tapering towards the connection-side end of the syringe cylinder, which on the one hand simplifies the sliding-on of the end of the flexible tubing and on the other hand ensures a good sealing due to the increased surface pressing between the flexible tubing and the raised part of the ring. At the same time the syringe cylinder, Luer connection and ring are manufactured as plastic injection molded parts, be it as one part or several parts. This tapering ring may furthermore also form part of a locking connection onto which a connection piece may be slid or by way of which a protective cap may be fastened.

The one-piece design is to be particularly preferred with regard to the manufacturing costs, in particular with large batch numbers, wherein the tool construction becomes more complicated and thus more costly.

The ring is preferably formed conical and comprises a circumferential edge which forms the actual sealing edge between the flexible tubing and the ring. This edge does not need to be an edge in the geometric sense but may be rounded or also be formed by a comparatively narrow raised ring section.

In order to simplify the sliding of a flexible tubing section onto the cylinder section of the Luer connection, it is advantageous if the ring with its tapering end merges into the cylinder section without a step so that on sliding on the flexible tube this is quasi automatically widened and guided by the ring.

The ring is preferably completely arranged at the connection-side end of the cylinder so that it closes flush with the end-face of the cylinder section. Where appropriate yet further rings may be integrally formed behind this ring closing flush with the cylinder section.

The tool construction may be considerably simplified by a design with which the syringe cylinder together with the Luer connection are formed as one piece as a plastic injection molded part, and the cylinder section of the Luer connection with the ring are formed as one piece with the ring as a plastic injection molded part. The two components after injection are joined together, and specifically connected to one another with a material fit, preferably by welding. With regard to tooling technology such a design is advantageous since the individual tools make do without any undercuts, thus are constructed relatively simply. This design is particularly advantageous in combination with a tamper-evident closure since then specifically the cylinder section to be welded on the actual syringe cylinder by way of ultrasound or in another manner may simultaneously form part of the tamper-evident closure. Alternatively also only the ring may form part of the tamper-evident closure which is then to be connected to the cylinder section in a suitable manner. This too is effected preferably by welding.

The syringe cylinder according to the invention preferably consists of polyolefins, preferably polypropylene (PP), cycle-olefin polymers (COP) or other barrier plastics. These materials are particularly suitable for manufacturing the previously mentioned injection molded parts, they are also easily weldable.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

FIG. 1 is a longitudinal section of the connection-side end of a syringe cylinder with a Luer lock connection and ring according to the invention;

FIG. 2 is a longitudinal section of an embodiment wherein the cylinder section and the ring are designed as a separate component;

FIG. 3 is a longitudinal section of a third embodiment wherein the ring and closure cap are manufactured as one piece; and

FIG. 4 is a detail of FIG. 3 in an enlarged representation.

### DETAILED DESCRIPTION OF THE PRESENTLY PREFERRED EMBODIMENTS

The connection-side end of a syringe cylinder 1 shown in FIG. 1 whose other end may be closed off by way of a plunger in the manner known per se, merges flushly into a syringe connection which consists of a Luer connection which is surrounded by a cylinder section 3 with an inner thread 4, so that a Luer lock connection results. On the outer side of the cylinder section 3 there is arranged a ring 6 tapering to the connection-side end 5 which in the embodiment according to FIG. 1 is designed as one piece with the syringe cylinder 1, the Luer connection 2 and the cylinder section 3.

The ring 6 comprises a narrow, essentially cylindrical section 7 which may also be designed rounded or as an edge, and assumes a sealing function (to a flexible tubing described further below). From this cylindrical section 7 there extends a section 8 which tapers conically towards the end 5 and which ends at the end-face 9 of the cylinder section 3. At the same time the shaping is such that the ring 6 with its conically tapering section merges into the outer circumference of the cylinder section 3 in a flush manner, i.e. recedes radially to a circumference which corresponds to that of the cylinder section 3. According to the wall thickness the circumference of the conically tapering section 8 at the connection-side end 5 may also be smaller than that of the cylinder section 3 in the remaining region.

The ring 6 serves essentially for fastening an elastic flexible tubing directly at the end of the syringe cylinder 1. With this the cross sectional tapering of the ring 6 towards the end 5 serves essentially for simplifying the sliding-on of a flexible tubing, whereas the section 7 is provided for achieving a good sealing effect between the flexible tubing and the connection. In this region one achieves the greatest surface pressing between the elastically widened flexible tubing and the ring 6 which as far as this is concerned is to be seen as being rigid, and thus achieves the sealing effect.

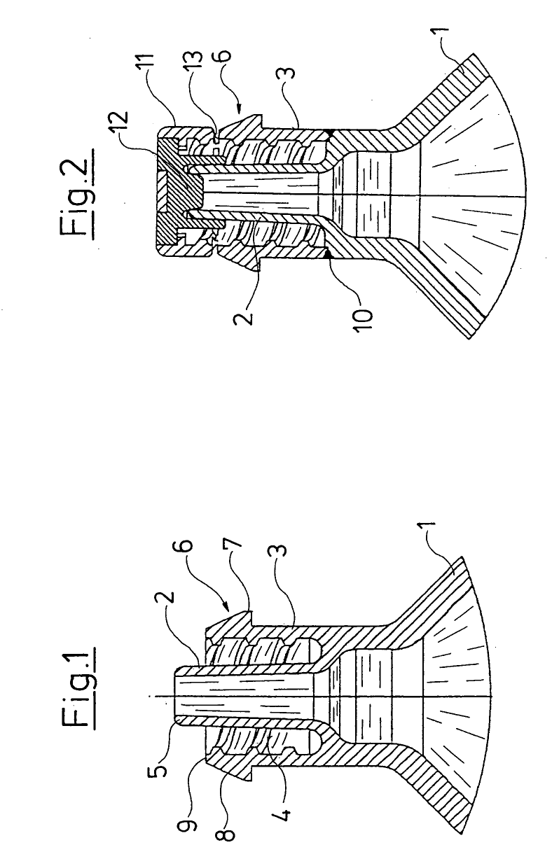

Although a syringe cylinder as has been described previously by way of FIG. 1 may be manufactured as one piece, the tool construction has a certain complexity due to the undercut present on the inner side as well as the outer side of the cylinder section 3, and this complexity may be avoided if, as shown by way of FIG. 2, the syringe cylinder 1 and the Luer connection 2 are manufactured as an injection molded part on the one hand and the cylinder section 3 with the ring 6 as a further, separate injection molded part of plastic on the other hand. The components are then connected to one another with a material fit, for example by a circumferential weld seam 10.

With the embodiment variants shown by way of FIG. 2 a closure cap 11 is further integrally formed onto the cylinder section 3 with the ring. A closure plug 12 is integrated with a positive fit or where appropriate injected into this closure cap 12, and encompasses the free end of the Luer connection on the inner side as well as the outer side and thus seals this off. Between the closure cap 11 and the cylinder section 3 there are formed thin webs 13 which form a frangible location for a later tamper-evident closure.

The injection molded part formed by the cylinder section 3 with the ring and the closure cap 11 after fitting in the closure plug 12 is placed onto the Luer connection 2 or onto the end of the syringe cylinder 1 in the position shown in FIG. 2 and is circumferentially welded so that the weld seam 10 is formed. The syringe cylinder 1 is then filled and sealingly closed by a plunger. For use, the frangible webs 13 are broken so that the cap 11 together with the closure plug 12 located therein can be removed. The syringe cylinder 1 opened in such a manner is then available for connection to the Luer connection 2, where appropriate in the form of a Luer lock connection whilst using the inner thread 4 as well as alternatively also as a flexible tubing connection over the outer circumference of the cylinder section 3 with a ring 36 located thereon.

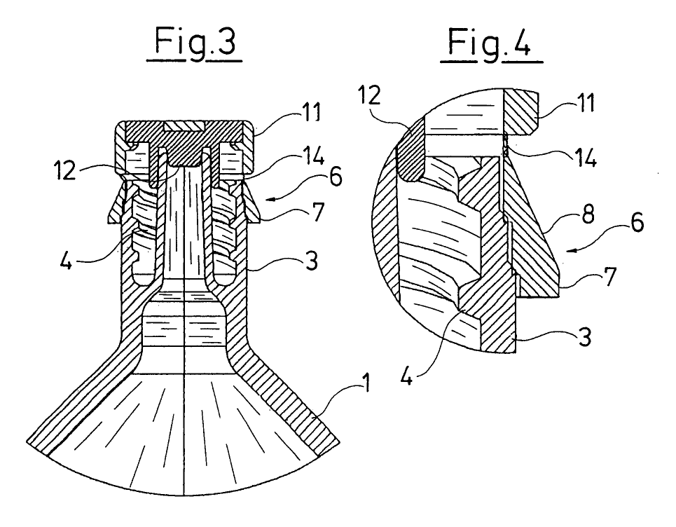

The design according to FIG. 3 differs from that previously described essentially in the fact that the syringe cylinder 1, the Luer connection 2 and cylinder section 3 are formed as a one-piece injection molded component on the one hand, and the ring 6 as well as the closure cap 11 and where appropriate molded closure plug 12 are formed as an injection molded part on the other hand. Between the closure cap 11 and the ring 6 there is provided a thin wall which form a frangible location for a tamper-evident closure as with the webs 13 in FIG. 2.

In the region of the ring 6 the outer sides of the cylinder section 3 as well as the inner side of the ring in each case are formed stepped essentially complementary to one another (see FIG. 4), wherein the ring 6 is pressed onto the free end of the cylinder section 3 so that this is held with a non-positive fit. One may alternatively also provide a welding here in order to connect these components to one another with a material fit. The function corresponds essentially to that described previously by way of FIG. 2.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed is:

1. A syringe cylinder comprising a syringe connection, said syringe connection comprising:
a Luer connection;
a cylinder section concentric to said Luer connection, said cylinder section comprising an inner thread, an outer circumference, and an end face; and
a ring on said outer circumference which tapers toward said end face.

2. A syringe cylinder as in claim 1 wherein said ring is formed as one piece with said cylinder section.

3. A syringe cylinder as in claim 1 wherein said ring has an outer circumferential edge and tapers conically from said edge toward said end face.

4. A syringe cylinder as in claim 1 wherein said ring tapers toward said end face in a stepless manner.

5. A syringe cylinder as in claim 4 wherein the ring ends flush with said end face.

6. A syringe cylinder as in claim 1 wherein said Luer connection and said cylinder section with said ring are formed as separate injection molded parts which are welded together.

7. A syringe cylinder as in claim 1 wherein said Luer connection and said cylinder are formed as a one piece injection molded part, and said ring is connected to said cylinder section by one of a material and a non-positive fit.

8. A syringe cylinder as in claim 7 wherein said ring is connected to said cylinder section by welding.

9. A syringe cylinder as in claim 1 further comprising a closure cap which is connected to one of said cylinder section and said ring by at least one frangible web to form a tamper-evident closure.

10. A syringe cylinder as in claim 1 wherein said cylinder consists of polyolefins.

11. A syringe cylinder as in claim 12 wherein said polyolefins comprise at least one of polypropylene and cyclo-olefin polymers.
